(19) United States
(12) Patent Application Publication
(10) Pub. No.: US 2008/0213725 A1
(43) Pub. Date: Sep. 4, 2008

(54) Method and Arrangement for a Dental Installation

(75) Inventors: Fredrik Adilstam, Lindingo (SE); Mattias Iverhed, Knivsta (SE)

Correspondence Address: KNOBBE MARTENS OLSON & BEAR LLP, 2040 MAIN STREET, FOURTEENTH FLOOR, IRVINE, CA, 92614, US

(73) Assignee: NOBEL BIOCARE AB (PUBL), Goteborg (SE)

(21) Appl. No.: 10/573,534

(22) PCT Filed: Sep. 7, 2004
(86) PCT No.: PCT/SE2004/001279

§ 371 Date: Apr. 15, 2008

(30) Foreign Application Priority Data
Sep. 24, 2003 (SE) 0302539-2

(51) Int. Cl. A61C 13/083; A61L 27/30

(52) U.S. Cl. 433/201.1; 427/2.27

(57) ABSTRACT

A substrate has or is provided on a surface with a first porosity. A dispersion with low-viscosity liquid is applied to the surface in order to form a ceramic layer with a second porosity having preferably larger and/or more pores than in the first porosity. The liquid has the ability to be sucked by capillary force into the first pore formation and, in a first stage, to retain on the surface material and/or liquid particles which do not penetrate into this first pore formation, and which contribute to the continued construction of the layer. In a second stage, the substrate is subjected to sintering in which the particles finally forming the layer are held together with intermediate spaces which consist of or are included in the second porosity, the spaces being formed either by the fact that material and liquid particles separate from the particles finally forming the layer are driven off during the sintering and/or by the fact that the particles forming the layer are chosen with a particle size which means that the last-mentioned particles are held together after the sintering despite the intermediate spaces. The invention also relates to an arrangement. By means of the invention, a dental product can be provided with excellent porosity which is advantageous in connection with dental installations.

The present invention relates inter alia to a method for a ceramic substrate which is included in or forms a unit which can consist, for example, of an implant, spacer, crown, etc., in a dental installation. The invention also relates to an arrangement in the form of a ceramic substrate of said type.

It is already known in dentistry to produce implants, crowns, spacers, etc., made of titanium with porous surface layers which have advantages when applied in or to bone substance, in dental installations, etc. It has been proposed that the porous surface layers be arranged mainly on structures made of titanium, and reference may be made in this connection, inter alia, to patents SE 514202 and SE 516282 obtained by the same Applicant as is filing the present patent application.

Reference is also made to the technique which is now generally known on the open market where the products provided by the same Applicant go under the trade name TiUnite, and which indicate possibilities of creating and using layers with excellent porosity on titanium components in dentistry. Examples which may be mentioned are crater-like structures with porosities of the order of 2-15 μm, for example 3-6 μm. The porosity can be used as a depot for different kinds of substances, for example growth-stimulating substances, anti-inflammatory substances, etc. The porosity per se is also advantageous for the actual incorporation of the implant in the jaw bone.

There is a particular need for ceramic products to be able to be produced with the same excellent porosity on the surfaces, for example on implant surfaces and spacer surfaces, and also on threads of the implant or spacer. However, there have been serious technical difficulties in being able to obtain porosities on ceramic products which, correspond to those found on metal products and which, for example, are intended to withstand the forces and moments which are formed or arise, for example, when screwing implants into holes formed in the jaw bone. In the case of crowns, for example, there is also a need to use cement to obtain considerable retention or fixing between the actual components of the installation. The present invention has the object of solving these problems, among others.

It is also important to be able to vary the thicknesses of the porous layers and their extents across the actual surface and the composition of the actual dental product. It is also desirable to be able to vary the degree of porosity and the distribution of the pores forming the porosity across the extent of the respective surface. The invention provides a solution to this problem too.

It should be noted that it is generally already known, outside the field of dentistry, to provide ceramic substrates with porous outer layers. However, it has been found that the technique used outside the field of dentistry cannot be transposed to the dental field without extensive rethinking, mainly on account of the demand for high porosity and precision in dentistry. Thus, for example, it is already known to produce porous ceramic layers in which ZrO₂ is used with Y₂O₃ in casting processes. Three different particles can be used to form pores, namely graphite, PMMA and NiO particles. The first two types of particles can be easily burnt off, while NiO has to be leached with acid. Reference may be made to the article “Synthesis of highly porous yttria-stabilized zirconia by tape casting methods”.

Another technique used outside the dental field is to form porous ceramic products with starch compositions, so-called starch forming. In these compositions, use is made of the possibility that the starch filled with water and moisture swells when it is heated above a certain temperature. The swelling forces the formation of a porous network, which creates a certain stability in the material. When the material is burned, a porous body is obtained. The starch particles can also be used as pore formers in accordance with the above. This method relates mainly to porosities extending completely through the product, which in general are not relevant in dentistry. The method is described in, inter alia, J. Am. Ceram. Soc. 86[3] 395-400 (2003); and “Processing of porous ceramics by a new direct consolidation technique”, J. Eur. Ceram. Soc. 1998, 18, 131-140.

A completely different technique used outside the dental field for the purpose of forming porous material would be to use zirconium parts with a large particle size. The greater the particle size, the larger the space obtained between the particles. A subsequent sintering thus will not be able to eliminate all pores when the ceramic particles are chosen with a large size. The porosity in the substrate preferably has a smaller size and the sintering will therefore eliminate the last-mentioned porosity.

That which can principally be regarded as characterizing a method according to the invention is, inter alia, that the substrate, at least at a portion bearing a surface, is provided with a first porosity, and that, in order to form a ceramic layer with a second porosity having pore sizes and/or pore numbers preferably exceeding the first porosity, a dispersion (suspension) of a preferably low-viscosity liquid is applied to the surface, said liquid having the ability to be sucked by capillary force into the first porosity or pore formation and, in a first stage, to retain on or against the surface material and/or liquid particles which do not penetrate into this first porosity and which contribute to the continued construction of the layer. The invention is also characterized in that, in a second stage, the substrate is subjected to sintering in which the particles finally forming the layer are held together with intermediate spaces which consist of or are included in the second porosity, the spaces being formed either by the fact that material and liquid particles separate from the particles finally forming the layer are driven off during the sintering and/or by the fact that the particles forming the layer are chosen with a particle size which means that the last-mentioned particles are held together after the sintering despite the intermediate spaces.

In one embodiment of the inventive concept, the particles are allocated a size and/or shape determining the pore formation, and the particles thus forming as pore formers are chosen to be or are insoluble in the liquid included in the dispersion. The particles for forming the dispersion are dispersible in the liquid with or without dispersants, and the particles can be easily driven off, by means of a removal function, for example burning in a furnace and/or etching and/or leaching and/or smelting and/or sublimation and/or dissolving. The particles are arranged or chosen to show a low residual degree of impurity after the removal function has been performed.

In a proposed embodiment, use is made of polycrystalline zirconia, alumina and/or hydroxyapatite mixed into the dispersion. As pore formers, it is possible to use particles of, for example, graphite with sizes in the range of 0.1-100 μm, preferably in the range of 0.3-50 μm. The range of 0.5-10 μm is of special interest. In addition, or alternatively, starch with particle sizes in corresponding ranges can be used. Said graphite and starch particles thus form the particles which are separate from the particles forming the layer. The substrate can be presintered to form the first porosity, and an emulsion used can be an acrylic polymer emulsion with liquid particles which are driven off in said second stage. In a further alternative, particles, for example of zirconia, alumina and/or hydroxyapatite can be admixed with the dispersion, these having a particle size which, after sintering, gives residual porosity. Water-soluble ammonium sulfate or polycarboxylate can be used for example as dispersant. Water and/or alcohol for example can be used as the low-viscosity liquid. The thickness, extent and composition of the layer can be varied within wide limits. Further embodiments are set out in the dependent claims relating to the novel method.

That which can principally be regarded as characterizing an arrangement according to the invention is that the substrate/arrangement, at least at a portion bearing a surface, has the ability to exhibit in an initial stage a first porosity or pore formation, and in that the surface bears a ceramic layer applied by means, inter alia, of sintering and with a second porosity having larger and/or more pores than in the first porosity. The last-mentioned porosity is arranged, before sintering of the layer, to have the ability to receive, by capillary force, a low-viscosity liquid and at the surface to cause the retention of particles dispersed in the liquid which contribute to the formation of the layer. In addition, the arrangement is characterized in that the construction of the layer is based on driving off by means of sintering the particles forming intermediate spaces, or in that the particles which form the layer have a particle size permitting the formation of intermediate spaces despite the sintering. In one embodiment, the last-mentioned particles can be of the same type as the substrate.

By means of what has been proposed above, dental products can be produced. The products in question can, for layer application, be immersed in a dispersion and, after the immersion, the product/blank/structure can be worked in order to remove excess. After drying, the coated cylinders or implants can be sintered with the layers thus applied. Even threaded implants can be coated and treated in a corresponding manner. This shows that said presintering gives advantageous results with good layer coating. The presintering can take place, for example, at 1200° C., for, for example, 2 hours before the layer coating is started. In the case of spacers and crowns, for example, the second porosity increases the base for cementing intended to hold the components in the installation together.

Presently proposed embodiments of a method and an arrangement according to the invention will be described below with reference to the attached drawings, in which:

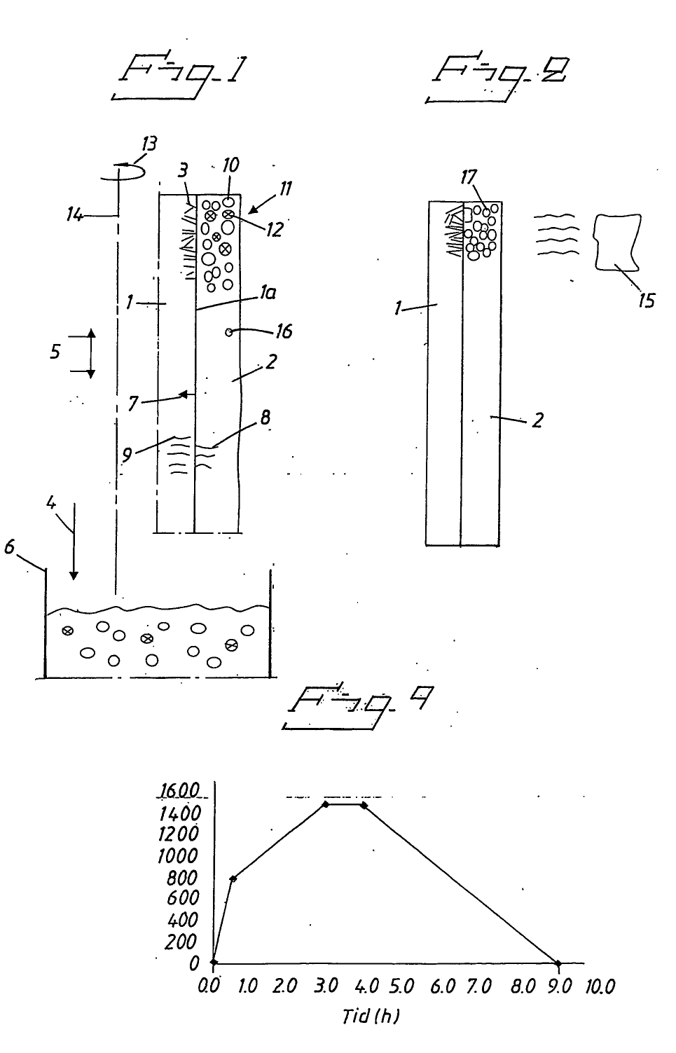

FIG. 1 shows symbolically, in a vertical view, parts of a unit consisting of an implant, crown, spacer, etc., coated with a layer of high porosity,

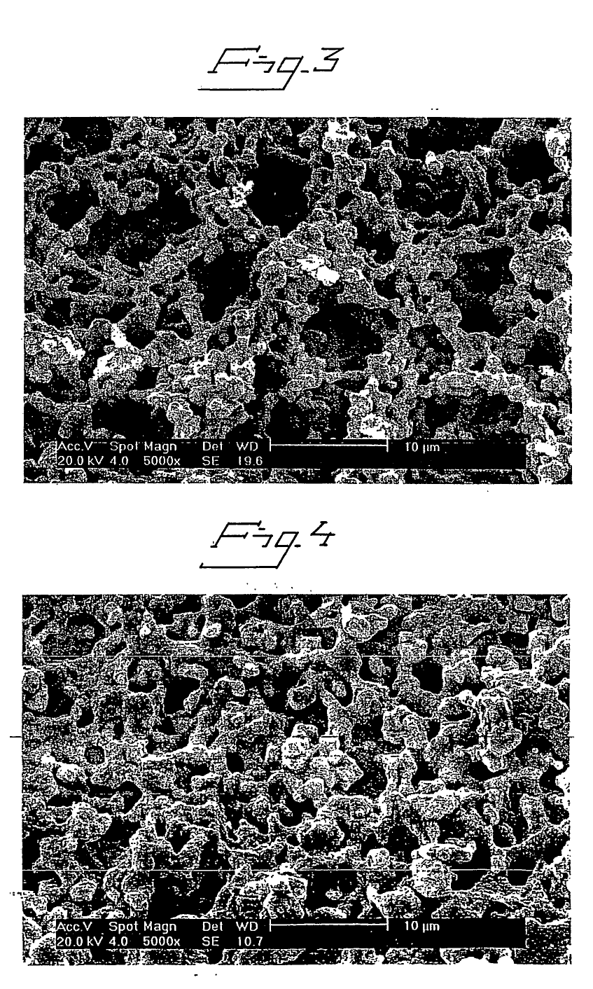

FIG. 2 shows symbolically, in a vertical view, the sintering method on the implant with associated layer,

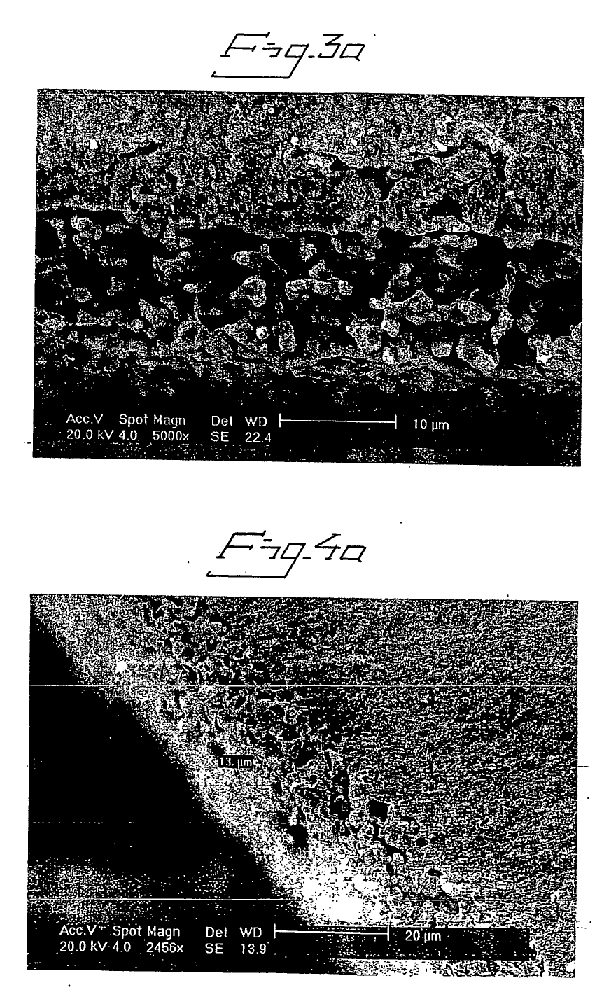

FIGS. 3-8a show different constructions of porous layers which have been produced by different methods, and

FIG. 9 shows, in diagram form, a sintering cycle in a furnace.

In FIG. 1, a unit is shown symbolically by reference number 1. According to the above, the unit can consist, for example, of an implant known per se, a spacer known per se, etc. The unit has a surface 1a which is to be coated completely or partially with a layer 2 of ceramic material of high porosity. The implant or equivalent is pre-sintered and has a first porosity 3 at least at said surface 1a, i.e. the implant can have a continuous porosity or it can have a densely sintered core with an outer layer formation which has said first porosity. On application of the layer 2, a dispersion 4 is used which is applied to said outer surface 1a by immersing the unit 1, see arrow 5, in a bath 6 of said dispersion. The application can alternatively be carried out by dripping, spraying, etc. The application means that a capillary force, which is indicated symbolically by 7, can come into play. The capillary force is generated by or with the first porosity 3 and means that liquid 8 is sucked into the porosity completely or partially. Thus, in the figure, liquid sucked into the porosity 3 is indicated by 9. The liquid 8 can penetrate with a complete degree of saturation into the porosity 3 or with a partial degree of saturation. The capillary force 7 means that the particles 10 and 12 which form layer 11 are retained on the surface 1a. The dispersion contains first particles 10 which are to form the final layer indicated by 17. In addition, particles 12 are included which can be driven off by subsequent sintering. In connection with the application, the unit can be rotated in the direction of the arrow 13 about its longitudinal axis 14. A characteristic of the particles 10 and 12 is that they are of such a size that they cannot penetrate to any great extent, or do not penetrate at all, into the first porosity 3.

FIG. 2 shows, symbolically, the sintering of the layer 2 with a sintering unit indicated symbolically by 15. In this sintering method, both the liquid 9 and the removable particles 12 are driven off. As an alternative, liquid particles 16 in an emulsion can be included instead of or in combination with the particles 12, which liquid particles can be driven off or burnt off similarly to the particles 12. In FIG. 2, the pore formation is indicated by 17.

The different pore formers produced different porosities, as can be seen from FIGS. 3-8a which show porous surfaces on ceramic implants.

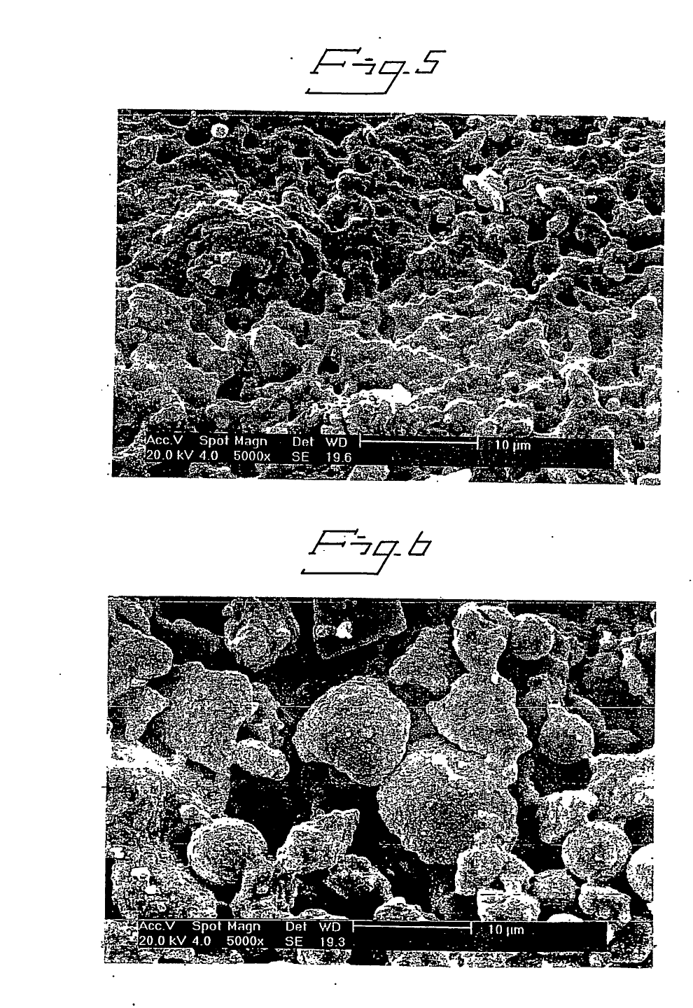

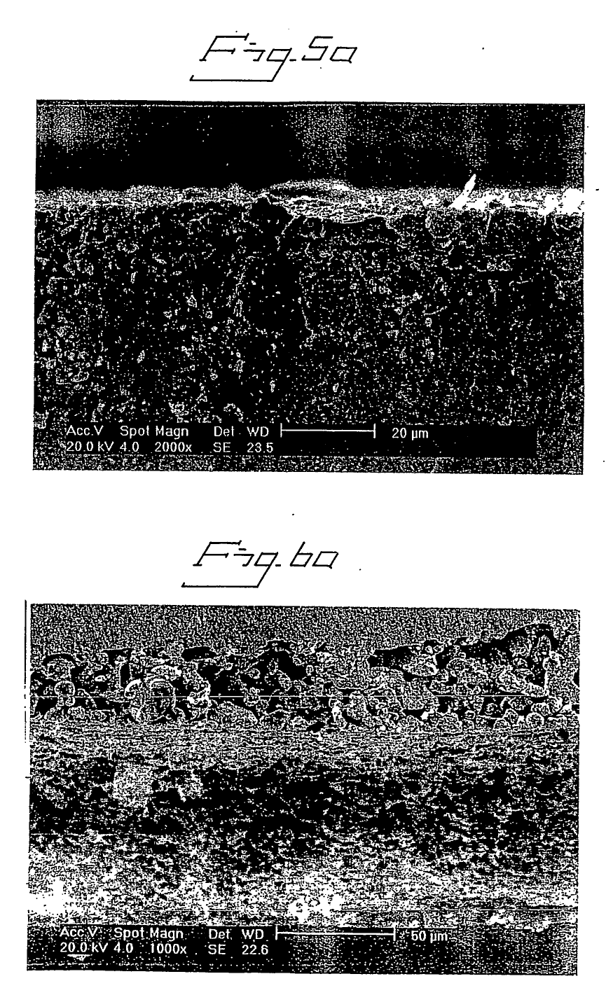

FIGS. 3-3b show a slurry with 50% by volume of graphite particles, and a particle content of 5% by volume. FIGS. 4-4a show a slurry with 50% starch particle and a particle content of 5%. FIGS. 5-5a show the case with 50% binder in the slurry and with a particle content of 5%. FIGS. 6 and 6a show coarse particles, with a particle content of 10% by volume. FIGS. 3, 4, 5 and 6 show the views at right angles to the surface, and 3a, 4a, 5a and 6a show the views at right angles to the fracture surface. Measurements have been carried out on the different layers and it appears that a reduction of the particle content increases the layer thickness.

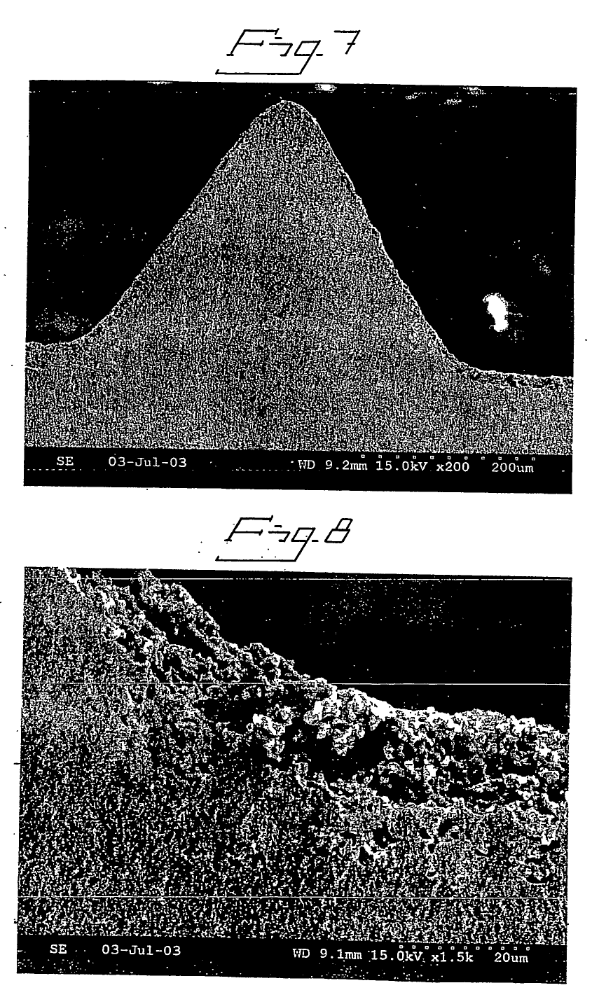

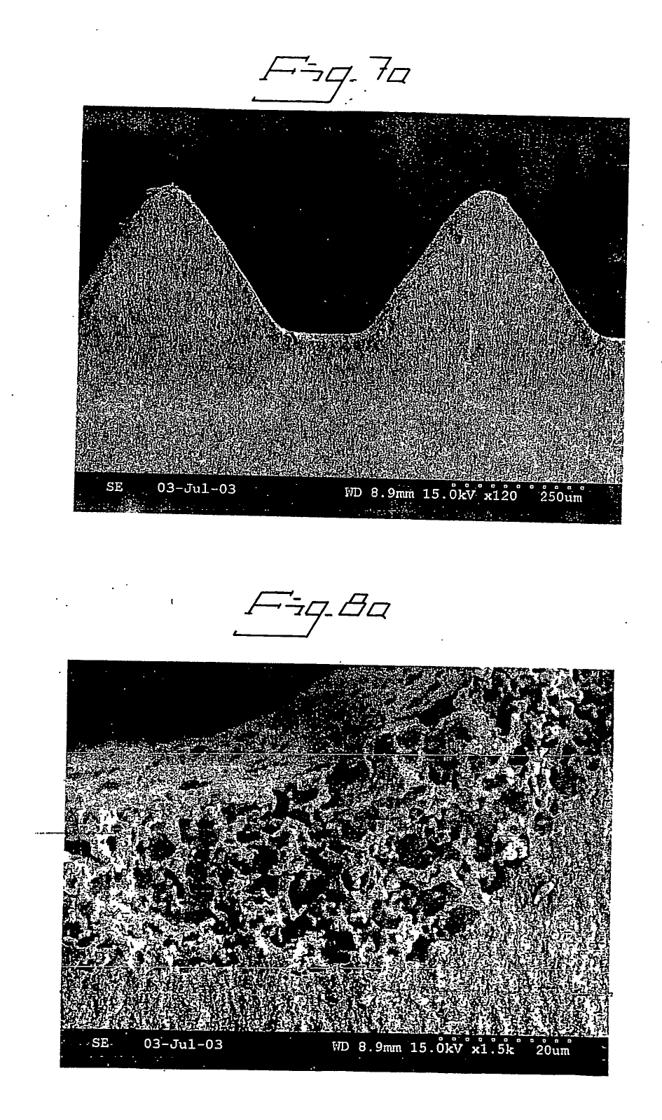

FIGS. 7-8a show cases of layer formation on a threaded implant. FIGS. 7 and 8 show the use of 50% by volume of graphite as pore former, and the figures show the use of starch as pore former. FIGS. 8 and 8a are on a larger scale than 7 and 7a.

The components can be sintered in the production and the implants in a furnace. A sintering cycle is shown in FIG. 9.

Components according to the invention have been produced and tested in accordance with the following examples.

### EXAMPLE 1

Reference is made to FIGS. 3 and 3a. A presintered, porous substrate in the form of a cylinder is produced from a commercially available yttrium-stabilized zirconia (Tosoh TZ-3YS-E). The implant was immersed in a dispersion which consisted of zirconia, a pore former of graphite, a dispersant and de-ionized water. The zirconium particles (TZ-3YS-E from Tosoh Corporation) had a mean particle size of ca. 0.3 μm. The graphite particles had a particle size of between 1 and 6 μm. The dispersant used was Duramax B3005 from Rohm and Haas which is a water-soluble ammonium salt of a polycarboxylate. The content of dispersant was 0.5 percent by weight. The dry content used was 5 percent by volume, the content of graphite particles in turn being 50 percent by volume.

After the immersion, the cylinder was rotated at 4500 rpm so that the excess of dispersion was removed from its surface. Subsequent sintering at 1500° C. and for 2 hours in an air atmosphere gave a porous layer with a thickness of ca. 20 μm.

### EXAMPLE 2

Reference is made to FIGS. 4 and 4a. A cylinder which in accordance with Example 1 has been immersed in a dispersion consisting of zirconia, a pore former of starch, a dispersant and de-ionized water was used. The zirconia particles in the dispersion corresponded to those used in Example 1. The starch particles had a particle size of between 3 and 6 μm (Remy DR, Remy, Belgium). The dispersant in Example 1 was used. The dry content used in the dispersion was 5 percent by volume, and the starch content was 50 percent by volume of the dry substance.

Immersion and the sintering method according to Example 1 gave a porous layer with a thickness of ca. 13 μm, cf. FIGS. 4 and 4a.

### EXAMPLE 3

Reference is made to FIGS. 5 and 5a. A cylinder corresponding to that in the example was immersed in a dispersion consisting of zirconia, a binder emulsion, a dispersant and de-ionized water. The zirconium particles in the dispersion corresponded to that used in Example 1. The emulsion was an acrylic polymer emulsion from Rohm and Haas, Duramax B1000. The dry content here was 5 percent by volume, and the binder content was 50 percent by volume of the dry substance.

Immersion and the sintering method according to Example 1 gave a porous layer with a thickness of ca. 15 μm.

### EXAMPLE 4

Reference is made to FIGS. 6 and 6a. A cylinder corresponding to the one in Example 1 was immersed in a dispersion consisting of zirconia particles, a dispersant and de-ionized water. The zirconium particles in the dispersion were coarser than those used in the production of the substrate, approximate particle size of between 7 and 10 μm. The dispersant used in Example 1 was also used here. The dry content here was 10 percent by volume.

Immersion and the sintering method according to Example 1 gave a porous layer with a thickness of ca. 40 μm.

### EXAMPLE 5

Reference is made to FIGS. 7 and 7a. A pre-sintered (1000° C.), porous substrate in the form of a threaded implant was made from a commercially available yttrium-stabilized zirconia (Tosoh TZ-3YS-E). The implant was immersed in a dispersion which consisted of zirconia, a pore former of graphite, a dispersant and de-ionized water. The zirconium particles (TZ-3YS-E from Tosoh Corporation) had a mean particle size of ca. 0.3 μm. The graphite particles had a particle size of between 1 and 6 μm. The dispersant used was Duramax B3005 from Rohm and Haas which is a water-soluble ammonium salt of a polycarboxylate. The dispersant content was 0.5 percent by weight. The dry content used was 5 percent by volume, the content of graphite particles in turn being 50 percent by volume.

After the immersion, the threaded implant was rotated at 4500 rpm so that the excess of dispersion was removed from its surface. Subsequent sintering at 1500° C. and for 2 hours in an air atmosphere gave a porous layer with varying thickness; in the thread parts the thickness was ca. 30 μm, while the layer thickness at the thread crests was only a few micrometers. Between top and bottom, the thickness was ca. 5 μm. Compare also FIG. 9.

### EXAMPLE 6

Reference is made to FIGS. 8 and 8a. An implant according to Example 5 was immersed in a dispersion consisting of zirconia, a pore former of starch, a dispersant and de-ionized water. Zirconium particles and dispersant according to Example 1 were also used here. The starch particles corresponded to those in Example 2. The dry content used in the dispersion was 5 percent by volume, and the starch content was 50 percent by volume of the dry substance.

The immersion and sintering method according to Example 1 gave a porous layer with a thickness of ca. 25 μm in the thread parts, while the crests had a thickness of ca. 5 μm.

A modification of the above method is to use, for formation of a ceramic layer, a substrate whose surface is of such a nature that it lacks the ability to suck liquid up by capillary force. Such a surface can be obtained, for example, by sintering of the abovementioned pre-sintered substrates. Application of the earlier suspension, with graphite as pore former for example, can be carried out, for example, by dripping, spraying and/or immersion. Since in these cases it is not possible to use a capacity of the liquid to penetrate in order to create the ceramic layer, the liquid phase in the dispersion must be removed in some other way, for example by drying.

The invention is not limited to what has been described above by way of example, and instead modifications can be made within the scope of the attached patent claims and the inventive concept.

1. A method for providing a porous surface layer on a ceramic substrate which forms a part of a a dental installation, comprising:
providing the ceramic substrate with, a surface with a first porosity;
forming a ceramic layer with a second porosity having larger and/or more pores than in the first porosity; the step of forming the ceramic layer, comprising:
applying a dispersion with a viscous liquid the surface, said viscous liquid having the ability to be sucked by capillary force into a first pore formation in the ceramic substrate to retain on the surface material and/or liquid particles of the dispersion which do not penetrate into the first pore formation and which contribute to the construction of the ceramic layer, and
sintering the dispersion to form the ceramic layer in which the particles finally forming the ceramic layer are held together with intermediate spaces which are included in the second porosity, the intermediate spaces being formed either by material and/or liquid particles separate from the particles finally forming the layer that are driven off during the sintering and/or by particles forming the layer that are chosen with a particle size that is sufficiently large such that the particles are held together after the sintering despite the intermediate spaces.

2. The method as in claim 1, further comprising allocating a size and/or shape of the particles that determines the pore formation, in that the particles are insoluble in the liquid included in the dispersion and are dispersible in the liquid with or without dispersant and can be driven off by a removal function comprising burning in a furnace, etching, leaching, smelting, sublimation and/or dissolving, and arranging the particles to show a low residual degree of impurity after the removal function has been performed.

3. The method as in patent claim 1, further comprising pre-sintering the ceramic substrate to form the first porosity, and mixing ceramic particles in the form of zirconia, alumina and/or hydroxyapatite into the dispersion, and assigning the ceramic particles sizes in the range of 0.1-1.0 μm.

4. The method as in claim 1, wherein the particles for pore formation can comprise graphite particles or starch particles and are assigned sizes in the range of 0.1-100 μm.

5. The method as in claim 1 comprising pre-sintering the substrate to form the first porosity, and forming the ceramic particles and the pore formers with an emulsion which comprises an acrylic polymer emulsion with liquid particles which can be driven off.

6. The method as in claim 1, comprising pre-sintering the substrate to form the first porosity, and mixing particles of zirconia, alumina or hydroxyapatite into the dispersion that have such a size that the porosity remains after sintering.

7. The method as in claim 1, wherein the thickness and/or extent of the layer on the surface is varied by one or more immersions in the dispersion and/or by variation of the dry substance content.

8. The method as in claim 1, wherein different pore formers are used to achieve the variation or variations in the pore structure of the layer.

9. The method as in claim 1, wherein the substrate is provided with a thread and the layer changes along the extent of the thread between the internal and external diameters.

10. The method as in claim 1, wherein the dispersion is applied to the surface by dripping, spraying and/or immersion.

11. The method as in claim 1, wherein water and/or alcohol is added to the viscous liquid.

12. A method for providing a porous ceramic layer on a ceramic substrate of a which forms a part of a a dental installation, comprising:
providing a a non porous surface,
applying a dispersion of a viscous liquid to the surface, said liquid having the ability to dry and to be retained surface material and/or liquid particles which do not penetrate into the non porous surface,
sintering such that the particles finally forming the layer are held together with intermediate spaces which are included in the porosity, the spaces being formed either by separating material and liquid particles separate from the particles finally forming the layer during the sintering and/or by forming the layer with particles that are chosen with a particle size are held together after the sintering despite the intermediate spaces.

13. A dental assembly comprising a ceramic substrate, wherein ceramic substrate, a first porosity and a ceramic layer applied to the ceramic substrate by sintering and with a second porosity having larger and/or more pores than in the first porosity, wherein first porosity is configured such that before sintering of the ceramic layer, to have the ability to receive, by capillary force, a low-viscosity liquid and at the surface retain particles dispersed in the liquid which contribute to the formation of the ceramic layer, and in that the increased porosity is formed by sintering of particles forming intermediate spaces, or in that the particles which form the layer have a particle size permitting the formation of intermediate spaces despite the sintering.
